(19) United States
(12) Patent Application Publication
(10) Pub. No.: US 2021/0252467 A1
(43) Pub. Date: Aug. 19, 2021

(54) NOVEL, LEAN AND ENVIRONMENT-FRIENDLY GRANULATION METHOD

(71) Applicant: Boehringer Ingelheim International GmbH, Ingelheim am Rhein (DE)

(72) Inventors: Hiroshi TAKASAKI, Hyogo (JP); Atsushi SAKURAI, Nishinomiya (JP); Takuma KATAYAMA, Kobe (JP)

(21) Appl. No.: 17/271,636

(22) PCT Filed: Aug. 27, 2019
(86) PCT No.: PCT/EP2019/072868

§ 371 Date: Feb. 26, 2021

(30) Foreign Application Priority Data
Aug. 30, 2018 (EP) 18 191 620.6
Jun. 26, 2019 (EP) 19 182 664.3

(51) Int. Cl. B01J 2/16; B01J 2/28; A61J 3/02

(52) U.S. Cl. CPC B01J 2/16; A61J 3/10; A61J 3/02; B01J 2/28

(57) ABSTRACT

An environment-friendly granulation process which can be used in pharmaceutical manufacturing and involves fluidized bed granulation of a pharmaceutical ingredient with a granulation liquid in the presence of a water absorbing substance.

### FIELD OF THE INVENTION

This invention relates to a novel fluidized bed granulation useful as a process step in particular in pharmaceutical manufacturing.

### BACKGROUND OF THE INVENTION

Granulation is a size enlargement process of converting fine particles into larger agglomerations, which is used, for instance, in pharmaceutical and food industry.

Wet granulation is one of the granulation techniques and widely used in the pharmaceutical industry as it provides easily flowing granules comprising a uniformly distributed active ingredient. It further masks potentially unfavorable compression properties and also equilibrates possible variabilities during drug product development or by sourcing active ingredients from different suppliers. In particular, high shear granulation (HSG) and fluidized bed granulation (FBG) are commonly used for this process (Faure et al., Eur. J. Pharm. Biopharm. 2001, 52, 269-277; Morin et al., AAPS PharmSciTech 2014, 15, 1039-1048).

A drawback of HSG in comparison to a direct compression or dry granulation process is that a further drying step is needed after the agglomeration stage in separate equipment, such as a tray dryer or fluidized-bed dryer. In addition, the formation of adhesions on the granulator walls and granule break-down by mechanical stress are process challenges during the drying step.

FBG is performed within the same equipment from granulation to the drying process. This can save transfer loss and reduce the operator exposure to irritating and/or toxic substances. As the FBG granulator is a low shear device compared to the high shear granulator, the granules are less likely to break down during the process. This is also contributing to the high porosity of the granules. Particle growth of FBG takes place as atomized binder droplets hit the fluidized granules, which enables uniform distribution of the binder. However, a fluidized bed granulator is initially expensive and requires the optimization of many parameters as well as a long spraying time for this granulation method. Also, granulation and drying proceed successively which may be time consuming.

In addition to potentially increased thermal stressing of the product induced by the microwave energy used for drying in HSG and FBG, both granulations cause higher manufacturing costs compared to the direct compression and dry granulation.

Therefore, moisture-activated dry granulation (MADG) may be an interesting and alternative way for a simple manufacturing process. MADG was initially described by Ullah et al. (Pharm. Technol. 1987, 11, 48-54; Pharm. Technol. 2009, 33, 42-51; Pharm. Technol. 2009, 33, 62-70). The entire process can be completed within a conventional high-shear granulator, with initial pre-blending of all components intended for granulation and a final blending with further functional excipients, such as disintegrants or lubricants just prior to compression. Therefore, a transfer of granule intermediates to other equipment in between process steps is avoided like in FBG, which can save processing time.

MADG can be divided into two stages: the agglomeration stage and the moisture absorption stage. Initially, the active pharmaceutical ingredient (API), water soluble fillers, and binders are pre-mixed in the granulator, and the binder is then activated by a small amount of water to form granules. MADG typically needs significantly less granulation liquid (mass ratio below 5% (m/m) with respect to the mass of the final blend without added water, e.g. 1-4% (m/m)) compared to the conventional HSG process. During the absorption stage, the moisture within the granules is reduced and distributed uniformly throughout the blend by subsequent addition of a water-insoluble filler-component, such as an absorbent powder.

A high shear granulator is mainly used for MADG because it allows for appropriate mechanical shear for granulation and mixing as well as water spraying function. As the granules experience mechanical force during the granulation process, the granules produced with a high shear granulator are denser than those produced with a fluidized bed granulator. A fluidized bed granulator, in contrast, is desirable for producing porous granules for achieving fast disintegration. Additionally, a fluidized bed granulator has the benefit of spraying granulation water uniformly.

### BRIEF DESCRIPTION OF THE DRAWINGS

FIG. 1: Manufacturing flow of GFBG with regard to equipment and materials used as well as operations performed

FIG. 2A: SEM image of FBG Granules according to Examples A and B

FIG. 2B: SEM image of GFBG-1 Granules according to Examples A and B

FIG. 3: Tensile strength of tablets as a function of compression force for GFBG-1, MADG-1, FBG and HSG (mean of n=10, SD)

FIG. 4: Disintegration time of tablets as a function of tensile strength for GFBG-1, MADG-1, FBG and HSG (mean of n=6, min/max)

FIG. 5: Porosity of tablets from GFBG-1 and FBG (mean of n=10, SD)

FIG. 6A: Initial wetting of tablets of approx. 3 MPa tensile strength from GFBG-1 and FBG (mean of n=3, SD)

FIG. 6B: Capillary wetting of tablets of approx. 3 MPa tensile strength from GFBG-1 and FBG (mean of n=3, SD)

FIG. 7A: Tensile strength of tablets as a function of compression force for MADG-2 with different amounts of water used for granulation (mean of n=10, SD)

FIG. 7B: Tensile strength of tablets as a function of compression force for GFBG-2 with different amounts of water used for granulation (mean of n=10, SD)

FIG. 8A: Disintegration profiles of tablets as a function of tensile strength for MADG-2 with different amounts of water used for granulation (mean of n=6, min/max)

FIG. 8B: Disintegration profiles of tablets as a function of tensile strength for GFBG-2 with different amounts of water used for granulation (mean of n=6, min/max)

### SUMMARY OF THE INVENTION

In a first aspect, the present invention relates to a fluidized bed granulation process comprising the subsequent steps of

a) transferring one or more ingredients into a fluidized bed granulator and mixing,

b) adding a suitable amount of granulation liquid to the fluidized powder bed by spraying and mixing,

c) adding a suitable amount of one or more moisture absorbents to the mixture and mixing under fluidized, and

d) optionally adding one or more further ingredients to the mixture, simultaneously or sequentially, and mixing after each simultaneous or sequential addition step,

wherein the inlet air temperature is below 60° C. throughout the process.

In a second aspect, the present invention relates to the use of a fluidized bed granulator for the process of the first aspect of the invention.

Further aspects of the present invention will become apparent to the person skilled in the art directly from the foregoing and following description and the examples.

### General Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The phrase “pharmaceutically acceptable” is employed herein to refer to those compounds, excipients, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

Pharmaceutically acceptable excipients suitable for the preparation of pharmaceutical dosage forms, e.g. solid oral dosage forms, will be known to those skilled in the art and comprise inert diluents, carriers, fillers, disintegrants, adjuvants, surfactants, binders, moisture absorbents, lubricants, sweeteners and/or colorants.

### DETAILED DESCRIPTION OF THE INVENTION

The present invention allows for a time-saving and environment-friendly granulation process, suitable e.g. for pharmaceutical manufacturing.

In a first aspect of the present invention, it is found that MADG concepts can be applied to FBG, leading to a process called green fluidized bed granulation (GFBG):

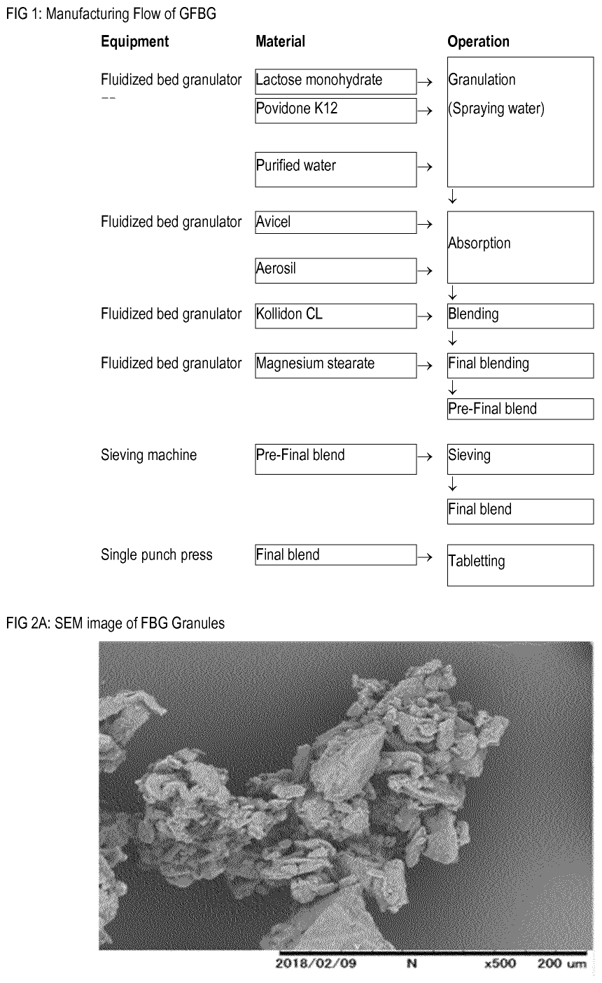

GFBG is based on MADG, i.e. it encompasses the stages of agglomeration and moisture absorption, wherein the entire process is performed in a fluidized bed granulator instead of a high shear granulator (see FIG. 1). GFBG consists only of the mixing, spraying and absorption process whereas the separate heating and drying steps of conventional FBG may be omitted. Also, unlike in conventional FBG, water may advantageously be used as a granulation liquid in GFBG such that the separate process step of preparation of a binder solution is not required.

In GFBG, like in MADG, granulation is effected by pre-mixing of ingredients, for instance of API and dry powder excipients, including water-soluble, nonabsorbent, easy-to-wet fillers, e.g. lactose monohydrate or mannitol, and/or binders, e.g. polyvidone, hydroxypropyl cellulose (HPC), copovidone, maltodextrins, maltose, sodium carboxy-methylcellulose (Na CMC), or hydroxypropyl methylcellulose (HPMC), and by subsequent spraying of granulation liquid, e.g. water or aqueous, alcoholic or hydroalcoholic binder solution, onto the mixture. In comparison to conventional wet granulation, only a significantly reduced amount of granulation liquid is required, usually a mass ratio of less than 10% (m/m) with respect to the mass of the final blend without added solvent, preferably 1-7% (m/m), more preferably 2-5% (m/m), e.g. 3% (m/m) or 4% (m/m). Further, moisture absorbents, such as microcrystalline cellulose and colloidal silicon dioxide, are added in order to reduce the moisture within the granules and to distribute it to the whole blend; the total mass fraction (m/m) of the absorbents may be more than 2.5% of the absorption mixture, preferably more than 10%, more preferably more than 20%. The upper limit of the absorbent mass fraction depends among others on the properties of the particular mixture and on the requirements for its further processibility, for instance into tablets, and may be e.g. 30%, 40%, 50%, 60%, 70%, 80% or 90% (m/m). The inlet air temperature during GFBG may be kept below 60° C., preferably below 50° C., more preferably below 40° C., most preferably in the range from 15° C. to 35° C., e.g. from 20° C. to 30° C., e.g. about 25° C. Thus, the temperature of the mixture within the granulator may be below 50° C., preferably below 40° C., more preferably below 30° C., most preferably in the range from 15° C. to 25° C., e.g. about 20° C., throughout the GFBG process. Depending, for instance, on the equipment used, the formulation applied and the batch size envisaged, the person skilled in the art will have no difficulty to derive the further parameters necessary to run the GFBG process successfully, e.g. spray droplet size, spraying surface and rate, inlet air flow rate, and process times, from the foregoing and following description as well as from his general knowledge.

The GFBG process according to the present invention has been explored in the context of tablet manufacturing with respect to manufacturability as well as granule and tablet properties in comparison with MADG, FBG and HSG. A first study was conducted exemplarily with the help of pharmaceutical placebo formulations PM, GFBG-1, MADG-1, FBG, and HSG, as described in Example A, each of which was manufactured in a 720 g batch size according to the processes of Example B:

With regard to manufacturability of the tablets, no problems were observed for any of the GFBG-1, MADG-1, FBG, and HSG manufacturing processes.

The GFBG-1 process time was well below 20 min for producing final blends, which was comparable with MADG-1; this process time is significantly shorter than that of FBG and HSG (see Example C). Importantly, the FBG process time cannot be further reduced due to the complex underlying granulation process. For instance, there are a number of key parameters for the FBG process, such as binder atomization, fluidization, wetting and spreading binder on the surface of the granules, agglomeration, consolidation, binder solidification and drying.

GFBG provides not only the shortest process time, but it also reduces the number of manufacturing machines compared to FBG and HSG (Example C).

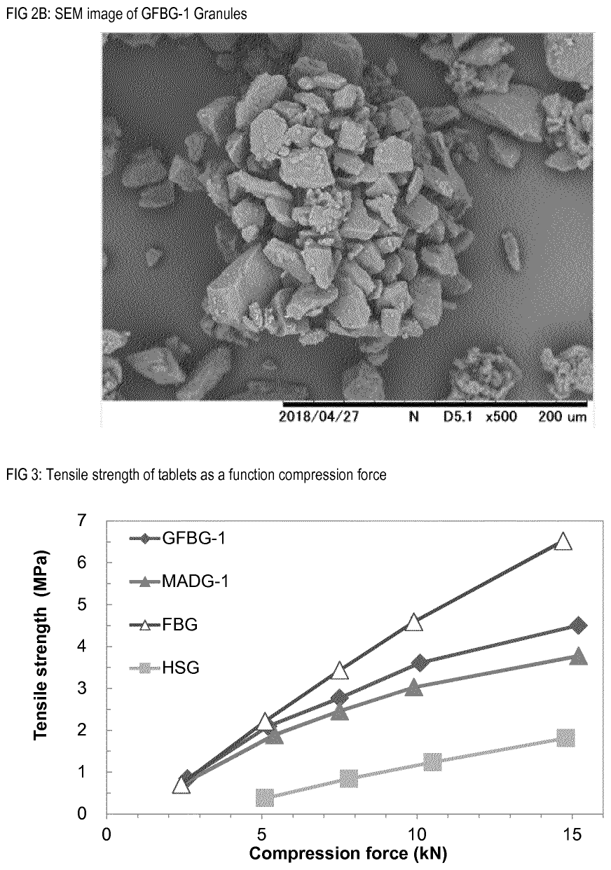

With regard to the granule properties (see Example D), the bulk density of the GFBG-1 granules was higher than the one of the FBG granules. This may have a positive influence on the granule flow and on the compressibility properties during tableting. Also, this may reduce the risk of problems during the tableting process for large size tablets since a significantly lower filling depth of the tableting can be employed for GFBG than for FBG (8.5 vs. 12.7 mm for the tablets of Example B). Of note, the GFBG-1 granules show a denser, more spherical appearance than the more loosely aggregated, more irregularly shaped FBG granules, as revealed by scanning electron microscopy (SEM) (FIG. 2A and FIG. 2B). Again, this morphology may positively impact the granule flow and the compressibility properties during tableting. The Hausner ratio for the GFBG-1 granules, being almost the same as for MADG-1, indicates in fact acceptable flowability. Accordingly, no problem is observed for the tablet mass variability during compression.

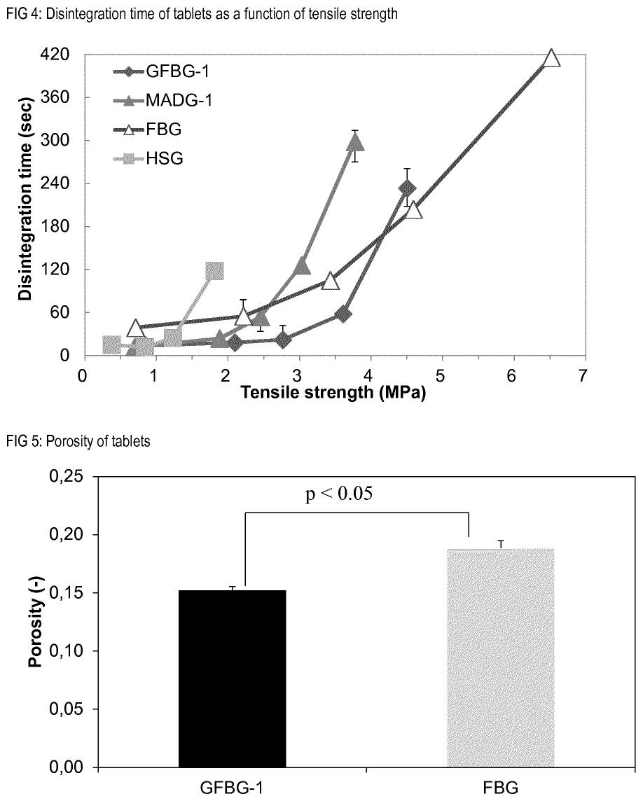

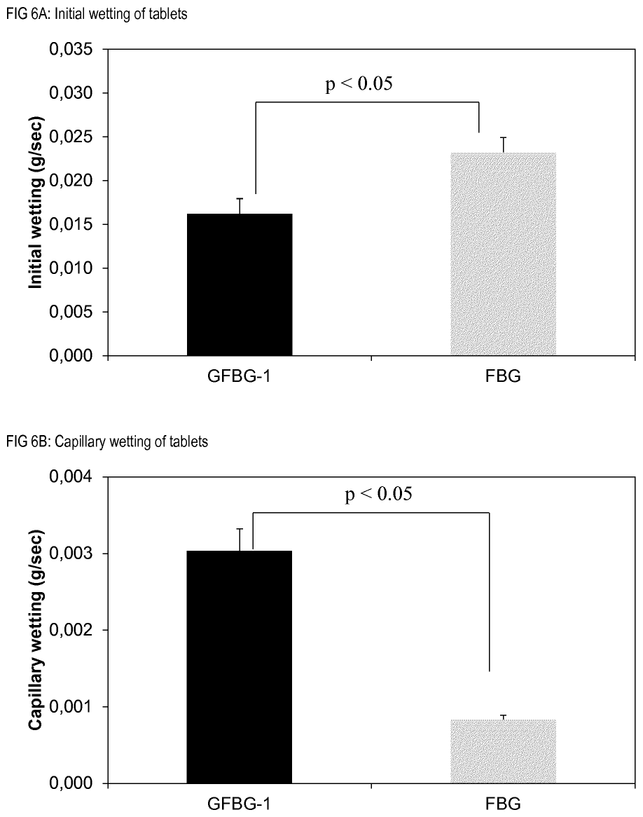

With regard to the tablet properties (see Example E), compression of GFBG-1 granules delivered tablets of similar tensile strength as MADG-1, as calculated from tablet hardness and dimensions. Sufficient tensile strengths (>1.5 MPa) were obtained for GFBG-1 tablets even at low compression forces (FIG. 3) while no tablets could be manufactured from the physical mixture (PM) due to poor granule flow and sticking during the tableting process. Furthermore, over a wide range of tensile strengths, GFBG-1 tablets showed the shortest disintegration times (FIG. 4) in comparison with tablets of comparable formulations and comparable tensile strengths obtained via the other manufacturing methods. Since tablet disintegration is related to wettability (initial and capillary wetting) and porosity, these parameters were investigated for GFBG-1 and FBG tablets of approximately 3 MPa tensile strengths. As a result, the GFBG-1 tablets showed slightly lower porosity (FIG. 5) and initial wetting (FIG. 6A). In contrast, the capillary wetting of the tablets with GFBG-1 was substantially (3.6 times) higher than of the tablets with FBG (FIG. 6B), possibly due to a loss of porosity of microcrystalline cellulose during the wet granulation process of FBG. This higher capillary wetting may have a positive impact on the disintegration time of the tablets obtained with GFBG-1.

In a second study, exemplarily, granules from pharmaceutical placebo formulations GFBG-2 and MADG-2 according to Example A were prepared with different amounts of water and were compressed into tablets, each according to the processes of Example B.

With regard to the granule properties (see Example D), the comparison of GFBG-2 and MADG-2 manufactured with the same amounts of water reveals lower loss on drying values and significantly lower water activities for the GFBG-2 granules, with the differences in water activity (an indicator of free water content) being higher than the differences in loss on drying. This is rationalized by the inlet air flow used in the GFBG process which may reduce the excess free water of the granules during the absorption process. Low water activities in solid drug products are generally advantageous because they are associated with a lower tendency towards microbial growth and a lower tendency towards hydrolytic degradation of moisture-sensitive active pharmaceutical ingredients. Also, high water activities may negatively impact physico-chemical parameters such as appearance, tablet hardness or dissolution.

Further, compressing the GFBG-2 and MADG-2 granules into tablets resulted in a lower tablet mass variability for GFBG-2 which is desirable e.g. in order to meet content uniformity criteria of a drug product.

The tablets, manufactured with different compression forces, were investigated with regard to their tensile strengths as a function of the amount of water used for granulation.

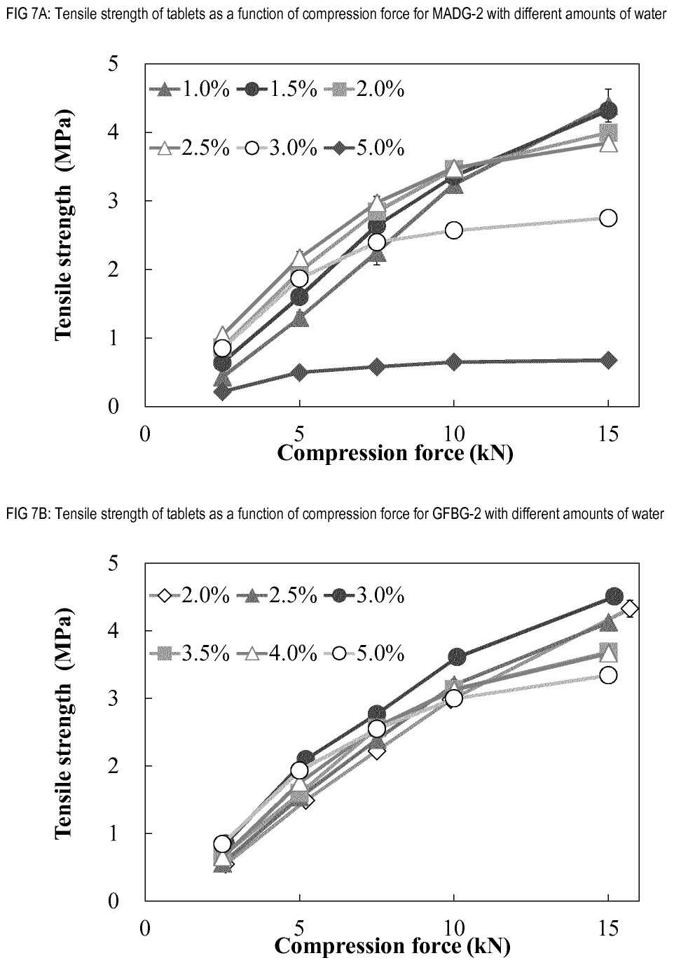

For MADG-2 tablets, a substantial decline in the tensile strength was observed already when a water mass ratio of 2.5% (m/m) was exceeded and no reasonable tablet tensile strength at all could be obtained from granules manufactured with 5.0% (m/m) of water (FIG. 7A). In contrast, for the GFBG-2 tablets, very good tensile strengths were observed over the whole water amount range from 2.0% (m/m) to 5.0% (m/m) with only a minor decrease for the highest water ratios at the highest compression force of 15 kN (FIG. 7B). The granulation of GFBG-2 thus might have a larger safety window with regard to the water amount than the granulation of MADG-2.

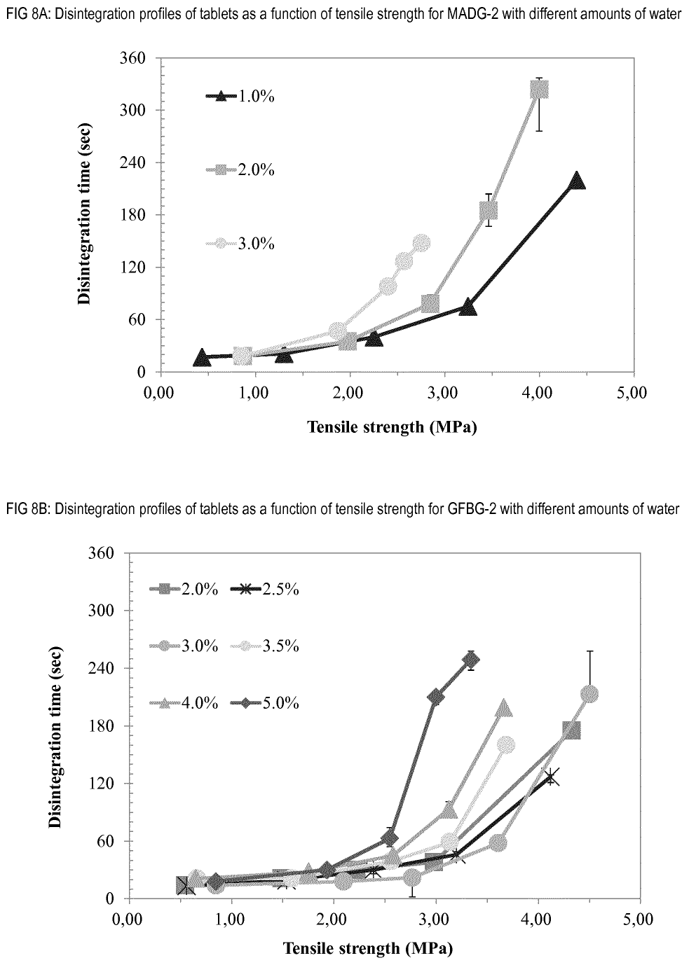

Also, the GFBG-2 tablets show shorter disintegration times (FIGS. 8A and 8B) than the corresponding MADG-2 tablets over a wide range of tensile strengths. Again, this may be explained by the substantially (2.4 times) higher capillary wetting of GFBG-2 tablets compared to MADG-2 tablets (see Example E, showing results for tablets with approx. 3 mPa tensile strength and a water activity of 0.5).

GFBG being free of the need for additional heating and drying steps thus provides a lean and environment-friendly granulation process that can be applied i.a. for the purposes of pharmaceutical manufacturing. For carrying out the complete process, only one piece of equipment, a fluidized bed granulator, is required, which saves processing time and keeps cleaning efforts as well as the risk of exposure to potentially hazardous compounds during transfers between process steps to a minimum. GFBG may hence be the ideal granulation process for the manufacturing of solid oral dosage forms of highly potent compounds.

Further, in the absence of a need for heating and drying, the fluidized bed granulator may be designed in a simpler and less expensive, more robust and less fault-prone manner. Further, not only is GFBG less energy consuming than current FBG or HSG methods, but also significantly shorter process times may be achieved. As a consequence, manufacturing costs are minimized. Also, potential stability issues of ingredients, in particular of APIs, due to exposure to heat, moisture and/or mechanical stress may be reduced. The properties of granules obtained with GFBG advantageously fulfill the criteria relevant, for instance, for processing into tablets, e.g. with regard to morphology, particle size distribution, flowability and density. Also their relatively low water activities may be advantageous. With regard to their processibility into tablets of acceptable mass variability, tensile strength and disintegration time, GFBG granules may exhibit a higher robustness and tolerance in respect of the amount of granulation liquid used, e.g. in comparison to MADG. The tablets obtained by compression of such granules may reveal favorable physico-chemical properties, such as fast disintegration, sufficient tensile strength even at low compression forces, low water activity, favorable porosity and wetting and low mass variability.

According to one embodiment of the first aspect of the present invention, a fluidized bed granulation process is provided comprising the subsequent steps of

a) transferring one or more ingredients into a fluidized bed granulator and mixing,

b) adding a suitable amount of granulation liquid to the fluidized powder bed by spraying and mixing,

c) adding a suitable amount of one or more moisture absorbents to the mixture and mixing, and

d) optionally adding one or more further ingredients to the mixture, simultaneously or sequentially, and mixing after each simultaneous or sequential addition step,

wherein the inlet air temperature is below 60° C. throughout the process.

According to another embodiment,

a fluidized bed granulation process is provided consisting of the subsequent steps a), b), c), and d).

According to another embodiment,

in step a) of the fluidized bed granulation process, the ingredients are selected from the group consisting of active pharmaceutical ingredients and pharmaceutically acceptable excipients,

preferably, the ingredients are one or more active pharmaceutical ingredients and one or more pharmaceutically acceptable excipients,

wherein, more preferably, the pharmaceutically acceptable excipients are selected from the group consisting of fillers, in particular water-soluble fillers, e.g. lactose monohydrate or mannitol, and/or binders, e.g. polyvidone, hydroxypropyl cellulose (HPC), copovidone, maltodextrins, maltose, sodium carboxymethylcellulose (Na CMC), or hydroxypropyl methylcellulose (HPMC).

According to another embodiment,

in step b) of the fluidized bed granulation process, the granulation liquid is selected from the group consisting of water and binder solution, preferably consisting of water and aqueous binder solution, more preferably it is water.

According to another embodiment,

in step b) of the fluidized bed granulation process, the amount of granulation liquid is below 10% (m/m), preferably 1-7% (m/m), more preferably 2-5% (m/m), e.g. 3% (m/m) or 4% (m/m).

According to another embodiment,

in step c) of the fluidized bed granulation process, the one or more moisture absorbents are selected from the group consisting of water-insoluble absorbents, e.g. microcrystalline cellulose and/or colloidal silicon dioxide.

According to another embodiment,

in step c) of the fluidized bed granulation process, the total amount of the one or more moisture absorbents is more than 2.5% (m/m), preferably more than 10% (m/m), more preferably more than 20% (m/m).

According to another embodiment,

in step d) of the fluidized bed granulation process, the further ingredients are selected from the group consisting of pharmaceutically acceptable excipients,

wherein preferably a first further pharmaceutically acceptable excipient is selected from the group consisting of disintegrants, e.g. crospovidone, and a second further pharmaceutically acceptable excipient is selected from the group consisting of lubricants, e.g. magnesium stearate.

According to another embodiment,

the inlet air temperature is preferably below 50° C., more preferably below 40° C., most preferably in the range from 15° C. to 35° C., e.g. from 20° C. to 30° C., e.g. about 25° C.

According to another embodiment,

in steps a), b), c) and d) of the fluidized bed granulation process, the temperature of the mixture within the granulator is below 50° C., preferably below 40° C., more preferably below 30° C., most preferably in the range from 15° C. to 25° C., e.g. about 20° C.

Further embodiments are described by the combination of any and each of the above definitions and embodiments with one another.

In a second aspect of the present invention, it is found that a fluidized bed granulator can advantageously be used for the process according to the first aspect of the invention, including its different embodiments.

### EXAMPLES AND EXPERIMENTAL DATA

The following examples are for the purpose of illustration of the invention only and are not intended in any way to limit the scope of the present invention.

A) Pharmaceutical Placebo Formulations

| | | | GFBG-1/-2 | | FBG |
|---|---|---|---|---|---|
| Process stage | Excipient | PM | MADG-1 | MADG-2 | HSG |
| Agglomeration | Lactose | 65.0 | 65.0 | 64.5 | 65.0 |
| | Monohydrate | | | | |
| | (Granulac 200) | | | | |
| | Iron oxide red | | | 0.1 | |
| | High-viscosity | — | — | | 5.0 |
| | Polyvidone | | | | |
| | (Povidone K25) | | | | |
| | Low-viscosity | 5.0 | 5.0 | 5.0 | — |
| | Polyvidone | | | | |
| | (Povidone K12) | | | | |
| | Microcrystalline | — | — | | 26.5 |
| | Cellulose | | | | |
| | (Avicel PH101) | | | | |
| Moisture | Microcrystalline | 26.4 | 26.4 | 26.4 | — |
| Absorption | Cellulose | | | | |
| | (Avicel | | | | |
| | PH102SCG) | | | | |
| | Colloidal Silicon | 0.1 | 0.1 | 0.5 | — |
| | Dioxide | | | | |
| | (Aerosil 200) | | | | |
| Main and Final | Crospovidone | 2.5 | 2.5 | 2.5 | 2.5 |
| Blending | (Kollidon CL) | | | | |
| | Magnesium | 1.0 | 1.0 | 1.0 | 1.0 |
| | stearate | | | | |
| | Total | 100.0 | 100.0 | 100.0 | 100.0 |

Excipient amounts in the above table are given as mass fractions.

Compositions PM, GFBG-1, MADG-1, FBG and HSG are manufactured in a batch size of 720 g each. The GFBG-2 formulation is manufactured in a batch size of 700 g. A batch size of 250 g is used for MADG-2.

B) Processes for Formulation Preparation

Preparation of PM (Physical Mixture)

Lactose monohydrate (Granulac 200, Meggle), polyvidone (Povidone K12, BASF), microcrystalline cellulose (Avicel PH102 SCG, FMC), colloidal silicon dioxide (Aerosil 200, Degussa), crospovidone (Kollidon CL, BASF) and magnesium stearate (Magnesium stearate vegetable, Faci) are blended for 10 min (Turbla mixer, T2F, Shinmaru Enterprises). The granules are used as the PM.

GFBG-1/-2 Process

GFBG-1 and GFBG-2 are processed in a fluidized bed granulator (MP-01, Powrex). The fluidizing air velocity is 0.3-0.4 m³/min, the inlet air temperature is 21° C. Fine grade lactose monohydrate is initially mixed with the binder Polyvidone in the fluidized bed granulator (1 min) and granulated by spraying water for 7 min (nozzle diameter 0.8 mm, atomizing air pressure 0.3 MPa, spray rate 3 g/min) into the granulator using a centered top spray nozzle. The granulation water amount is 3% (m/m) for GFBG-1, whereas amounts of 2.0%, 2.5%, 3.0%, 3.5%, 4.0% and 5.0% (m/m) are used for GFBG-2. For the 5 min absorption stage, the moisture absorbents microcrystalline cellulose and colloidal silicon dioxide are added. Finally, the disintegrant crospovidone and pre-sieved lubricant magnesium stearate are added directly into the granulator for 1.5 min and 0.5 min, respectively. The temperature of the mixture within the granulator is below 22° C. throughout the granulation process. The final blends are sieved by a conical sieving machine (1.0 mm rasp sieve, Quadro Comil U5, Powrex).

The manufacturing flow of GFBG is also depicted in FIG. 1.

MADG-1 Process

MADG-1 is processed in a high-shear granulator (Diosna P1/6, Diosna) equipped with a 4 L granulation bowl. Processing parameters are kept constant throughout the agglomeration (1 min) and massing (3 min) stages: impeller 500 rpm, chopper 1200 rpm. Fine grade lactose monohydrate is initially mixed with the binder Polyvidone, and granulated by spraying water for 15 s (nozzle diameter 0.3 mm, atomizing air pressure 2.5 bar) into the granulation bowl. The granulation water amount is 2% for MADG-1. For the 2 min absorption stage, the moisture absorbents microcrystalline cellulose and colloidal silicon dioxide are added when the chopper is stopped. Finally, the disintegrant crospovidone, and pre-sieved lubricant magnesium stearate are added directly into the granulator for 1.5 min and 0.5 min, respectively, with a reduced impeller speed of 250 rpm. The final blends are sieved by a conical sieving machine (1.0 mm rasp sieve, Quadro Comil U5, Powrex).

MADG-2 Process

MADG-2 is processed in a high-shear granulator (Diosna P1/6, Diosna) equipped with a 1 L granulation bowl. Processing parameters are kept constant throughout the agglomeration (1 min) and massing (3 min) stages: impeller 500 rpm, chopper 1200 rpm. Fine grade lactose monohydrate containing small quantities of iron oxide red (Univar Ltd.) is initially mixed with the binder Polyvidone, and granulated by spraying water for 15 s (nozzle diameter 0.3 mm, atomizing air pressure 2.5 bar) into the granulation bowl. The amounts of added water are 0.0%, 1.0%, 1.5%, 2.0%, 2.5%, 3.0%, and 5.0% (m/m). For the 2 min absorption stage, the moisture absorbents microcrystalline cellulose and colloidal silicon dioxide are added when the chopper is stopped. Finally, the disintegrant crospovidone, and pre-sieved lubricant magnesium stearate are blended directly into the granulator for 1.5 min and 0.5 min, respectively, with a reduced impeller speed of 250 rpm. The final blends are sieved by a conical sieving machine (1.0 mm rasp sieve, Quadro Comil U5, Powrex).

FBG Process

The binder Polyvidone (Povidone K25, BASF) is dissolved in water in a glass vessel with propeller mixer for 30 min. The solid content of binder solution was 10%. Fine grade lactose monohydrate and microcrystalline cellulose (Avicel PH101, FMC) are initially put in the fluidized bed granulator for 1 min and granulated by spraying binder solution (MP-01, Powrex: fluidizing air velocity 0.3-0.4 m³/min, inlet air temperature 75 C.°, nozzle diameter 0.8 mm, atomizing air pressure 0.3 MPa, spray rate 5 g/min) using a centered top spray nozzle. The wet granules are dried in a fluidized bed granulator (MP-01, Powrex: fluidizing air velocity 0.3-0.4 m³/min, inlet air temperature 75 C.°) and sieved by a conical sieving machine (1.0 mm rasp sieve, Quadro Comil U5, Powrex). The disintegrant crospovidone and pre-sieved lubricant magnesium stearate are finally blended with the granules for 5 min and 2 min, respectively (Turbla mixer, T2F, Shinmaru Enterprises).

HSG Process

Fine grade lactose monohydrate and microcrystalline cellulose are initially mixed with the binder Polyvidone and granulated by adding 20% (m/m) water. Wet granules are passed through a 2 mm screen. The wet granules are dried in a fluidized bed granulator (MP-01, Powrex: fluidizing air velocity 0.3-0.4 m³/min, inlet air temperature 75 C.°) and sieved by a conical sieving machine (1.0 mm rasp sieve, Quadro Comil U5, Powrex). The disintegrant crospovidone and pre-sieved lubricant magnesium stearate are finally blended with the granules for 5 min and 2 min, respectively

(Turbla mixer, T2F, Shinmaru Enterprises).

Preparation of Tablets

The final blends of the GFBG-1/-2, MADG-1/-2, HSG and FBG processes are compressed to flat faced tablets of 8 mm diameter and 200 mg mass on an eccentric press (Korsch EKO, Korsch) (FlexiTab, Manesty) at different compression forces of approximately 2.5 kN, 5.0 kN, 7.5 kN, 10.0 kN and 15.0 kN.

C) Process Parameters

Process time for producing final blends (720 g scale)

| Process Step | GFBG-1 | MADG-1 | FBG | HSG |
|---|---|---|---|---|
| Preparation of binder solution | — | — | 30 | — |
| Pre-heating machine | — | — | 5 | — |
| Granulation | 7 | 5 | 70 | 5 |
| Absorption | 5 | 2 | — | — |
| Wet sieving | — | — | — | 5 |
| Pre-heating machine | — | — | — | 5 |
| Drying | — | — | 9 | 22 |
| Sieving | — | — | 1 | 1 |
| Blending | 1.5 | 1.5 | 5 | 5 |
| Final blending | 0.5 | 0.5 | 2 | 2 |
| Sieving | 1 | 2 | — | — |
| Total (min) | 15 | 11 | 122 | 45 |

Number of machines for producing final blends

| Process | GFBG-1 | MADG-1 | FBG | HSG |
|---|---|---|---|---|
| Preparation of | — | — | propeller mixer | — |
| binder solution | | | | |
| Granulation | fluidized bed | high shear | fluidized bed | high shear |
| | granulator | granulator | granulator | granulator |
| Wet sieving | — | — | — | wet-sieving |
| | | | | machine |
| Drying | — | — | fluidized bed | fluidized bed |
| | | | granulator | granulator |
| Sieving | — | — | sieving | sieving |
| | | | machine | machine |
| Blending | fluidized bed | high shear | blender | blender |
| | granulator | granulator | | |
| Final blending | fluidized bed | high shear | blender | blender |
| | granulator | granulator | | |
| Sieving | sieving | sieving | — | — |
| | machine | machine | | |
| Total number | 2 | 2 | 4 | 5 |
| of machines | | | | |

D) Granule Properties

Particle Size Distribution

The particle size distributions of the final blends are measured by sieve analysis (Robot Shifter RPS-95, Seishin) employing the following screens: 500 μm, 355 μm, 250 μm, 180 μm, 125 μm, 90 μm, 75 μm, and 63 μm, fora sifting time of 5 min on vibration level 4 and pulse interval of 1 s (n=1). Interpolation nodes for d50 are iterated using the software Easysieve (Retsch).

Bulk Density, Tapped Density, Flowability

Bulk and tapped densities of the final blends are determined in a 100 mL sample cup on a powder property measurement system (Tapped density tester SVM121, ERWEKA) by applying 1250 taps (n=1). The Hausner ratio as a surrogate for flowability is calculated as the ratio of tapped and bulk density: (ρ tapped/ρ bulk). The flowability is classified according to US Pharmacopeial Convention benchmarks from excellent (1.00-1.11), good (1.12-1.18), fair (1.19-1.25), to passable (1.26-1.34).

Flow Time

The flow time is measured for a pre-weighed sample (100 g) using granule flow tester (GTB, ERWEKA).

Loss on Drying

Approximately 5 g of final blend is heated at 105° C. for 10 min using a moisture analyzer HG63-P (Mettler Toledo, Ohio) (n=1).

Water Activity

The water activity of the final blends is determined using a water activity measuring device (LabMaster-aw basic; Novasina, Switzerland). Approximately 5 g of the sample is warmed to 25° C. prior to measurement (n=1). The temperature is also kept constant during the measurement.

Scanning Electron Microscopy (SEM) Image

SEM images of the granules are taken using a Microscope TM3000 (Hitachi-hitech). The granules are mounted on the plate; the samples are coated by Au using a sputtering coating device (MSP-mini magnetron sputter, Shinkuu device).

Summary of Results

| Parameter | PM | GFBG-1 | MADG-1 | FBG | HSG |
|---|---|---|---|---|---|
| D50 (μm) | 55 | 100 | 95 | 110 | 107 |
| Bulk | 0.46 | 0.43 | 0.52 | 0.29 | 0.66 |
| density | | | | | |
| (g/mL) | | | | | |
| Hausner | 1.70 | 1.30 | 1.32 | 1.37 | 1.23 |
| ratio | | | | | |
| Flow time | no flow | 28.2 | 30.8 | 36.0 | 25.0 |
| (s/100 g) | | | | | |
| Tablet mass | — | 0.35 | 0.32 | 0.28 | 0.20 |
| variability | | | | | |
| (%) | | | | | |

| | GFBG-2/MADG-2 | | | |
|---|---|---|---|---|
| | 2.0% | 2.5% | 3.0% | 5.0% |
| Parameter | water | water | water | water |
| Loss on | 2.95/3.35 | 3.38/3.63 | 3.35/4.16 | 4.11/5.49 |
| drying (%) | | | | |
| Water | 0.41/0.56 | 0.46/0.61 | 0.49/0.66 | 0.58/0.78 |
| activity | | | | |
| Tablet mass | 0.32/0.50 | 0.19/0.43 | 0.35/0.95 | 0.22/0.80 |
| variability | | | | |
| (%) | | | | |

E) Tablet Properties

Hardness and Thickness

Hardness of the tablets and tablet thickness are measured using a tablet hardness tester (MultiTest 50, SOTAX) (n=10). Tensile strength is calculated as 2 F/(π*D*T): where F is the hardness, and D and T are the diameter and thickness of the tablets, respectively.

Disintegration Time

Disintegration time of the tablets is measured at 37° C. using a disintegration tester (NT-400, Toyama) at 30 cycles/min (n=6). No discs are added. The test medium is water.

Porosity

Tablet porosity (c) is calculated by employing the equation ε=1−(m/ρ true*V), where m and V are mass and volume of the tablets, respectively. True density (ρ true) is measured using a mercury penetration porosimeter (Amico) (n=1).

Wettability

Wettability measurement is performed at 25° C. with a surface tension balance (K21, KRUSS GmbH) in which the mass of the adsorbed liquid is measured versus time. For testing tablets, the tablet is put directly into the stainless tube. The stainless tube packed with moisture absorbent or tablet is lowered into water and a note of the time is made when the water contacts the stainless tube. The weight of the water that penetrated into the excipient or tablet is recorded against time. The data is collected every 20 ms. For evaluating wettability, initial wetting and capillary wetting (g/s) are calculated based on the profile. The wetting behavior is divided into two phenomena: initial wetting and capillary wetting. Initial wetting is defined as wetting of the tablet surface. After that, water penetrates into the tablet, which is defined as capillary wetting. The initial wetting is calculated by using first 5 points on linear regression, while capillary wetting is calculated by using linear regression at the equilibrium condition.

### Summary of Results

| Parameter | GFBG-2 | MADG-2 |
|---|---|---|
| Porosity | 0.1522 | 0.1353 |
| Initial wetting (g/sec) | 0.0162 | 0.0164 |
| Capillary wetting (g/sec) | 0.0030 | 0.0012 |

1-12. (canceled)

13. A fluidized bed granulation process comprising the subsequent steps of
(a) transferring one or more ingredients into a fluidized bed granulator and mixing,
(b) adding a suitable amount of granulation liquid to the fluidized powder bed by spraying and mixing,
(c) adding a suitable amount of one or more moisture absorbents to the mixture and mixing, and
(d) optionally adding one or more further ingredients to the mixture, simultaneously or sequentially, and mixing after each simultaneous or sequential addition step,
wherein the inlet air temperature is below 60° C. throughout the process.

14. The process according to claim 13, wherein in step (a) the ingredients are selected from the group consisting of active pharmaceutical ingredients and pharmaceutically acceptable excipients.

15. The process according to one or more of claim 13, wherein in step (a) the ingredients are one or more active pharmaceutical ingredients and one or more pharmaceutically acceptable excipients.

16. The process according to one or more of claim 14, wherein in step (a) the pharmaceutically acceptable excipients are selected from the group consisting of fillers and binders.

17. The process according to one or more of claim 13,
wherein in step (b) the granulation liquid is selected from the group consisting of water and binder solution.

18. The process according to one or more of claim 13,
wherein in step (b) the amount of granulation liquid is below 10% (m/m).

19. The process according to one or more of claim 13, wherein in step (c) the one or more moisture absorbents are selected from the group consisting of water-insoluble absorbents.

20. The process according to one or more of claim 13, wherein in step (c) the total amount of the one or more moisture absorbents is more than 2.5% (m/m).

21. The process according to one or more of claim 13, wherein in step (d) the further ingredients are selected from the group consisting of pharmaceutically acceptable excipients.

22. The process according to claim 21, wherein in step (d) a first further pharmaceutically acceptable excipient is selected from the group consisting of disintegrants and a second further pharmaceutically acceptable excipient is selected from the group consisting of lubricants.

23. The process according to one or more of claim 13, wherein the inlet air temperature is in the range from 15° C. to 35° C.

24. Use of a fluidized bed granulator for the process of one or more of claim 13.
